(19) United States
(12) Patent Application Publication
(10) Pub. No.: US 2018/0271449 A1
(43) Pub. Date: Sep. 27, 2018

(54) HEARTBEAT DETECTION SYSTEM AND HEARTBEAT DETECTION METHOD

(71) Applicant: KEIO UNIVERSITY, Tokyo (JP)

(72) Inventors: Tomoaki OTSUKI, Kanagawa (JP); Eriko Mogi, Kanagawa (JP)

(21) Appl. No.: 15/922,164

(22) Filed: Mar. 15, 2018

(30) Foreign Application Priority Data
Mar. 23, 2017 (JP) 2017-057779

(51) Int. Cl. A61B 5/00; A61B 8/08; A61B 5/024

(52) U.S. Cl. CPC A61B 5/726; A61B 5/02405; A61B 8/488

(57) ABSTRACT

A heartbeat detection system includes a Doppler sensor configured to receive a reflective wave from a subject, to obtain a Doppler signal; and a processor including a memory and a CPU. The processor executes applying a wavelet transform to the Doppler signal based on a plurality of scale factors, to obtain a wavelet coefficient for each of the scale factors; detecting a plurality of peaks of the wavelet coefficient for each of the scale factors; calculating each peak interval between the peaks next to each other, and each difference between the peak intervals next to each other, for each of the scale factors; selecting one of the scale factors that has a minimum variation in the differences of the peak intervals, as an optimal scale factor; and measuring the peak intervals calculated based on the optimal scale factor as heartbeat intervals.

### CROSS-REFERENCE TO RELATED APPLICATIONS

This application is based upon and claims the benefit of priority of Japanese Priority Application No. 2017-057779 filed on Mar. 23, 2017, the entire contents of which are hereby incorporated by reference.

### TECHNICAL FIELD

The present disclosure relates to a heartbeat detection system and a heartbeat detection method.

### BACKGROUND ART

Conventionally, technologies have been known that measure heartbeats by using a Doppler sensor and a wavelet transform. Among these technologies, a technology has been proposed that executes learning for selecting a scale factor for a wavelet transform.

As an example, there is a technology that obtains measurement data from a measuring device to measure motion of a human body; stores data that has been measured for the motion of the human body when a subject as a target of measurement is in a rest state, as reference data; determines a scale factor based on the reference data; and applies a wavelet transform to the measurement data, based on the determined scale factor to measure heartbeats (see, for example, Japanese Unexamined Patent Application Publication No. 2015-192715).

The above technology takes into consideration only the number of peaks of the wavelet coefficient as a criterion for selecting a scale factor; but even if the number of peaks continuously exhibits the same value, it does not necessarily correspond to responding to the heartbeat, and hence, there has been room for improvement in detection precision of heartbeat intervals (R-R intervals).

The present invention has been made in view of the above, and has an object to improve detection precision of heartbeat intervals.

### SUMMARY OF THE INVENTION

According to an embodiment, a heartbeat detection system includes a Doppler sensor configured to receive a reflective wave from a subject, to obtain a Doppler signal; and a processor including a memory and a CPU. The processor executes applying a wavelet transform to the Doppler signal based on a plurality of scale factors, to obtain a wavelet coefficient for each of the scale factors; detecting a plurality of peaks of the wavelet coefficient for each of the scale factors; calculating each peak interval between the peaks next to each other, and each difference between the peak intervals next to each other, for each of the scale factors; selecting one of the scale factors that has a minimum variation in the differences of the peak intervals, as an optimal scale factor; and measuring the peak intervals calculated based on the optimal scale factor as heartbeat intervals.

According to the disclosed technology, it is possible to improve detection precision of heartbeat intervals.

### BRIEF DESCRIPTION OF THE DRAWINGS

FIG. 1 is a diagram exemplifying a general configuration of a heartbeat detection system according to a first embodiment;

FIG. 2 is an example of a Doppler signal obtained by a Doppler sensor 10;

FIG. 3 is a diagram exemplifying hardware blocks of a signal processor according to the first embodiment;

FIG. 4 is a diagram exemplifying functional blocks of a signal processor according to the first embodiment;

FIG. 5 is an example of a flowchart illustrating operations of a heartbeat detection system according to the first embodiment;

FIG. 6 is an example of scale factors;

FIG. 7 is an example of a wavelet coefficient;

FIG. 8 is an example of frequency distribution of differences of calculated adjoining peak intervals;

FIG. 9 is an example of distribution of R-R intervals actually obtained with an electrocardiograph;

FIG. 10 is a diagram illustrating outliers in the frequency distribution illustrated in FIG. 8;

FIG. 11 is a diagram illustrating selection of an optimal scale factor;

FIG. 12 is an example of differences of adjoining peak intervals;

FIG. 13 is a diagram illustrating interpolation of peak intervals;

FIG. 14 is a diagram illustrating RMSEs of an application example and a comparative example;

FIG. 15 is a diagram comparing data before and after interpolation of peak intervals in an application example;

FIG. 16 is an example of a flowchart illustrating operations of a heartbeat detection system according to a second embodiment;

FIG. 17 is a diagram illustrating extraction of a template; and

FIG. 18 is an example of a correlation function of a template and a wavelet coefficient.

### DETAILED DESCRIPTION OF THE PREFERRED EMBODIMENTS

In the following, embodiments will be described with reference to the drawings. Note that throughout the drawings, the same components are assigned the same reference symbols, and duplicated description may be omitted.

### First Embodiment

FIG. 1 is a diagram exemplifying a general configuration of a heartbeat detection system according to a first embodiment. As illustrated in FIG. 1, the heartbeat detection system 1 has a Doppler sensor 10 and a signal processor 20 as main components.

The Doppler sensor 10 is a sensor that observes a frequency shift between a transmitted signal and a received signal generated by the Doppler effect, to detect motion of an observation target (a subject). In the present embodiment, a continuous wave (CW) is used as a wave to be transmitted, as an example.

The Doppler sensor 10 is placed near a subject, receives a signal (a reflective wave) reflected on the subject, to obtain a Doppler signal generated by a motion of the observation target. The subject may be, for example, a patient sleeping in a hospital or a nursing home, a driver of a vehicle, or the like.

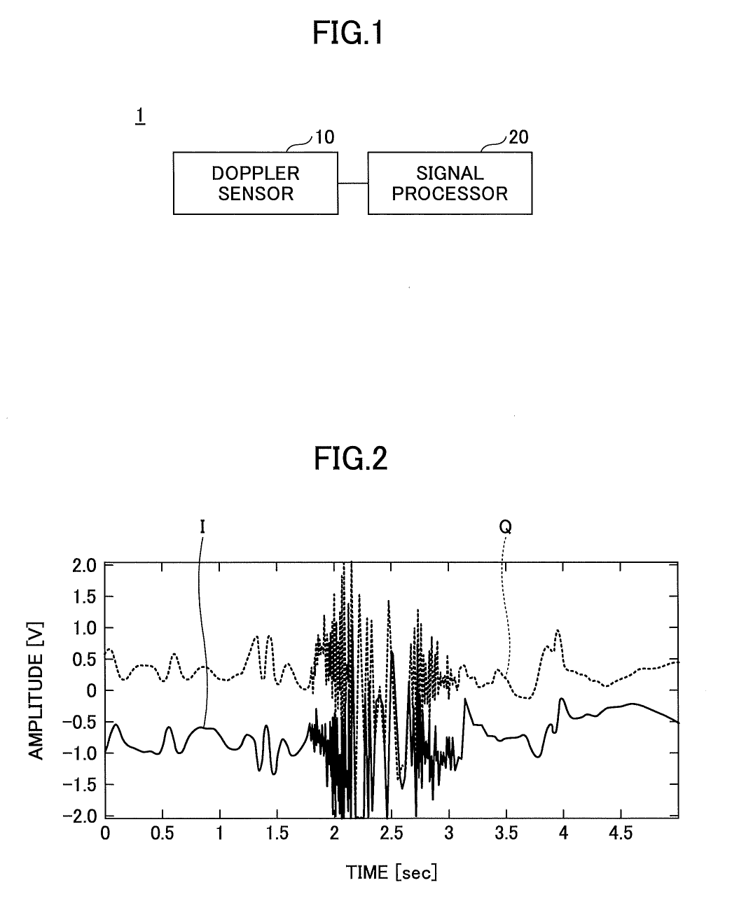

FIG. 2 is an example of a Doppler signal obtained by the Doppler sensor 10. The signal illustrated in FIG. 2 is a Doppler signal representing a frequency shift between a transmitted signal and a received signal as a function of time, which is constituted with an I signal being an in-phase component of the transmitted signal, and a Q signal being a quadrature component. It is favorable to remove noise appropriately by using a bandpass filter or the like depending on frequency components of a detection target.

Referring back to FIG. 1, the signal processor 20 detects the heartbeat of the subject, based on a Doppler signal, which is an output signal of the Doppler sensor 10. The signal processor 20 may properly use the I signal and the Q signal of a Doppler signal received by the Doppler sensor 10 as they are, or may generate various signals (amplitude, phase, integral values of these, etc.) based on the I signal and the Q signal.

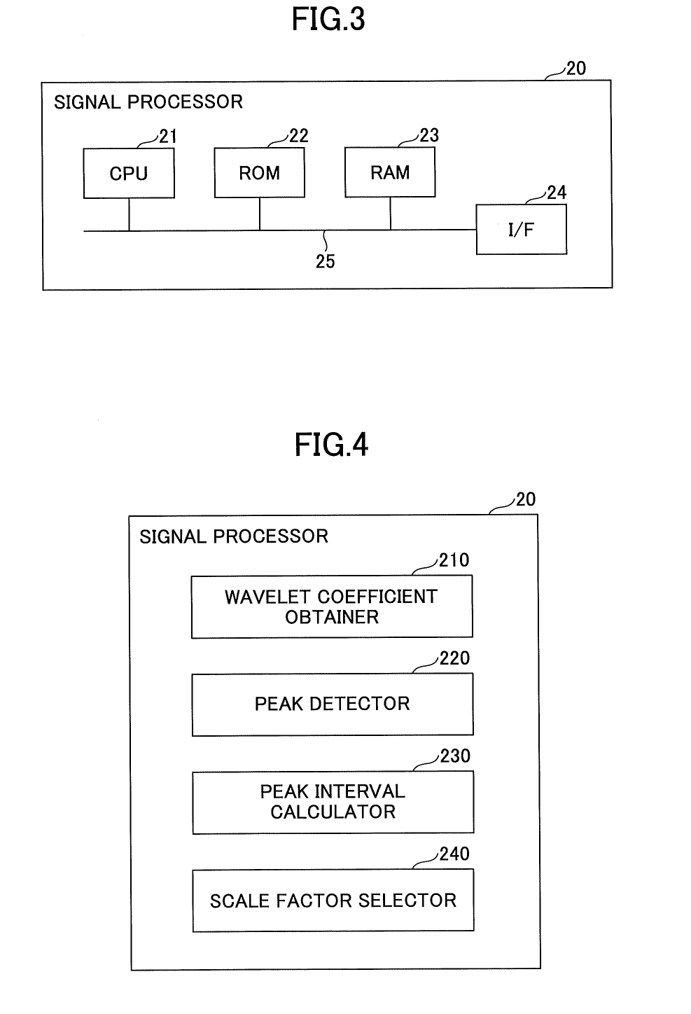

FIG. 3 is a diagram exemplifying hardware blocks of the signal processor 20 according to the first embodiment. Referring to FIG. 3, the signal processor 20 includes a CPU 21, a ROM 22, a RAM 23, an interface 24, and a bus line 25. The CPU 21, the ROM 22, the RAM 23, and the interface 24 are mutually connected through the bus line 25.

The CPU 21 controls functions of the signal processor 20. The ROM 22 as a storing means stores a program executed by the CPU 21 to control the functions of the signal processor 20, and various information items. The RAM 23 is a storing means used as a work area or the like of the CPU 21. The RAM 23 can also store predetermined information temporarily. The interface 24 is an interface for connecting the heartbeat detection system 1 with another device. The heartbeat detection system 1 may be connected to an external network or the like through the interface 24.

Here, the signal processor 20 may be a processor of general-purpose use, or a part or the entirety of the signal processor 20 may be implemented by application-specific hardware. Also, the signal processor 20 may be physically constituted with multiple devices and the like.

FIG. 4 is a diagram exemplifying functional blocks of the signal processor 20 according to the first embodiment. Referring to FIG. 4, the signal processor 20 includes, as functional blocks, a wavelet coefficient obtainer 210, a peak detector 220, a peak interval calculator 230, and a scale factor selector 240.

The wavelet coefficient obtainer 210 has a function to apply a wavelet transform to a Doppler signal based on multiple scale factors, to obtain a wavelet coefficient. The peak detector 220 has a function to detect peaks of a wavelet coefficient. The peak interval calculator 230 has a function to calculate each interval between peaks next to each other (a peak interval) of a wavelet coefficient, and as well, to calculate each difference between peak intervals next to each other (“peak intervals next to each other” may also be referred to as “adjoining peak intervals”, below). The scale factor selector 240 has a function to select an optimal scale factor, based on variation of differences of peak intervals of a wavelet coefficient from the center value.

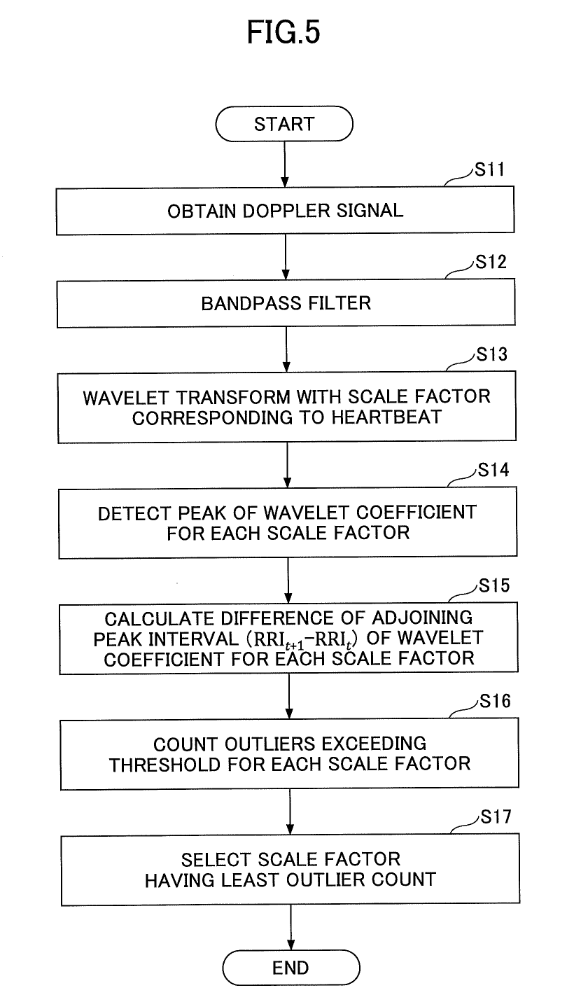

FIG. 5 is an example of a flowchart illustrating operations of the heartbeat detection system 1 according to the first embodiment. Referring to FIG. 5 mainly and the other figures appropriately, a heartbeat detection method will be described according to the first embodiment.

First, at Step S11, the Doppler sensor 10 obtains a reflective wave from a subject. Such a Doppler signal may be obtained by the Doppler sensor 10 placed in front of the chest of the subject separated by approximately 1 m while the subject is in a seated state. The Doppler signal obtained here is constituted with, for example, the I signal and the Q signal as illustrated in FIG. 2. Subsequent signal processing may be applied to each of the I signal and the Q signal; may be applied to the electric power of the I signal and the Q signal (I²+Q²); or may be applied to the amplitude of the I signal and the Q signal (√I²+Q²).

Next, at Step S12, the system removes noise components of the Doppler signal obtained at Step S11. Removal of noise components may be executed by hardware, for example, by inserting a bandpass filter between the Doppler sensor 10 and the signal processor 20. Alternatively, the removal may be executed by digital signal processing (by a digital filter or the like) in the signal processor 20 by directly inputting the output signal of the Doppler sensor 10 into the signal processor 20.

Although the passband of the bandpass filter can be selected properly depending on an environment, an observation target, and the like, since the frequency of heartbeat is approximately 0.5 to 1.2 Hz, the passband may be set, for example, greater than or equal to 0.5 Hz and less than or equal to 5 Hz.

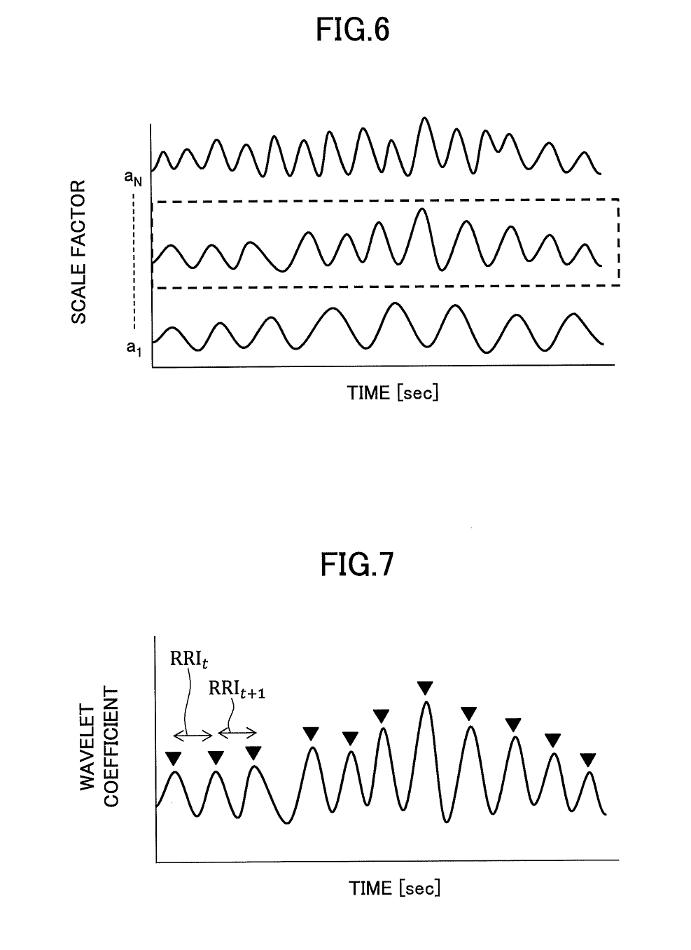

Next, at Step S13, the wavelet coefficient obtainer 210 executes a wavelet transform with multiple scale factors corresponding to heartbeats, to obtain a wavelet coefficient for each of the scale factors. FIG. 6 illustrates an example of N items of scale factors (where N is a natural number) of a₁ to a_{N}; and FIG. 7 illustrates an example of one of the wavelet coefficients among a₁ to a_{N}.

Here, a scale factor is a scaling rate when magnifying or reducing a mother wavelet (a fundamental waveform). In the heartbeat detection system 1, the frequency of the heartbeat being approximately 0.5 to 1.2 Hz, and a feature of R-R intervals (heartbeat intervals) that intervals next to each other in time do not change significantly, are taken into consideration to select in advance multiple scale factors corresponding to the heartbeat based on a statistic of R-R intervals, and to store the scale factors in the RAM 23. The number of scale factors stored in the RAM 23 in advance can be selected properly as necessary, which may be made, for example, several dozens. Also, the wavelet coefficient is a quantity that represents the strength of correlation with the mother wavelet.

Next, at Step S14, the peak detector 220 detects peaks of the wavelet coefficient for each of the scale factors. A peak of a wavelet coefficient can be detected by a known method that uses the second derivative or the third derivative. A peak of a wavelet coefficient may be detected by using another known method. Reverse triangular marks illustrated in FIG. 7 designate an example of detected peaks of a wavelet coefficient.

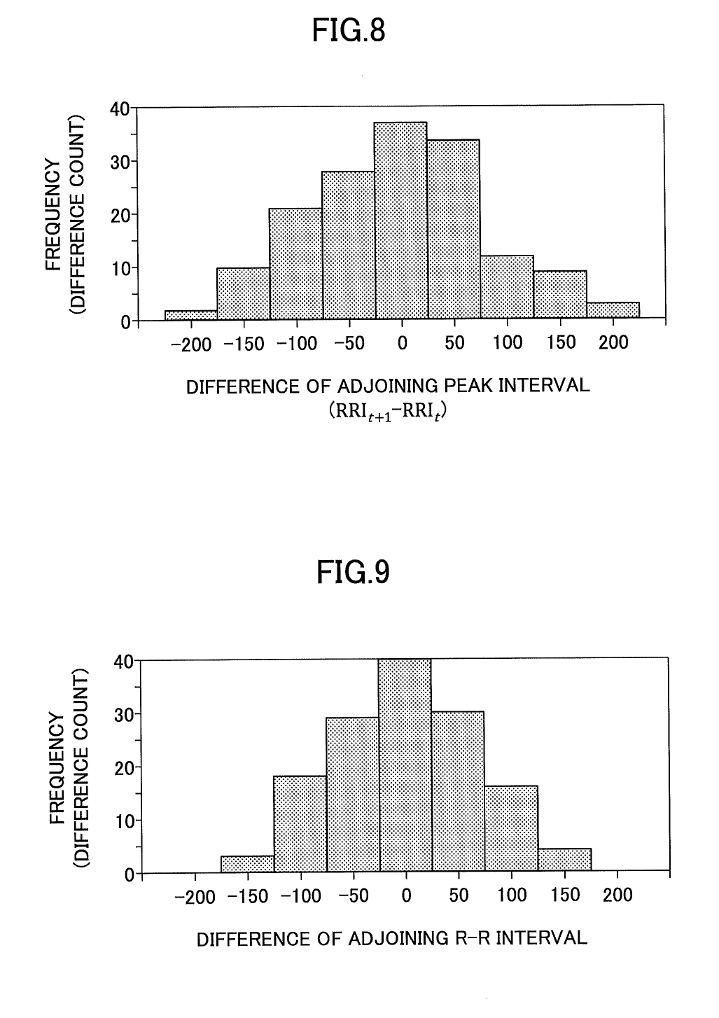

Next, at Step S15, the peak interval calculator 230 calculates each interval between peaks next to each other (a peak interval) for the peaks detected at Step S14, and further calculates each difference between peak intervals next to each other (adjoining peak intervals), for each of the scale factors. For example, in FIG. 7, in the case where the interval between the first and second peaks (a peak interval) is calculated as RRIₜ, and the interval between the second and third peaks (another peak interval) is calculated as RRIₜ₊₁, (RRIₜ₊₁−RRIₜ) is calculated as the difference between the adjoining peak intervals. This calculation is executed for all of the peaks one by one. FIG. 8 illustrates an example of frequency distribution of differences of calculated adjoining peak intervals. Frequency distribution of differences of adjoining peak intervals is calculated for each scale factor.

Next, at Step S16, the scale factor selector 240 compares, for each of the scale factors, the frequency distribution of differences of adjoining peak intervals calculated at Step S15, with a threshold value on the positive side and a threshold value on the negative side that have been determined in advance, to count the sum total of a frequency greater than or equal to the threshold value on the positive side, and a frequency less than or equal to the threshold value on the negative side, as the count of outliers.

Note that the threshold value on the positive side and the threshold value on the negative side may be determined, for example, based on distribution of R-R intervals actually obtained with an electrocardiograph, which may be stored in the RAM 23. FIG. 9 is an example of distribution of R-R intervals actually obtained with an electrocardiograph. In this case, since the frequency of differences of adjoining peak intervals exceeding ±200 is zero, the threshold values may be set, for example, to 200 on the positive side and to −200 on the negative side, to be stored in the RAM 23. Here, it is assumed in the following description that the threshold value on the positive side is equal to 200, and the threshold value on the negative side is equal to −200. However, the threshold value on the positive side and the threshold value on the negative side are not necessarily determined based on distribution of R-R intervals actually obtained with an electrocardiograph, and may be determined based on a statistic of R-R intervals.

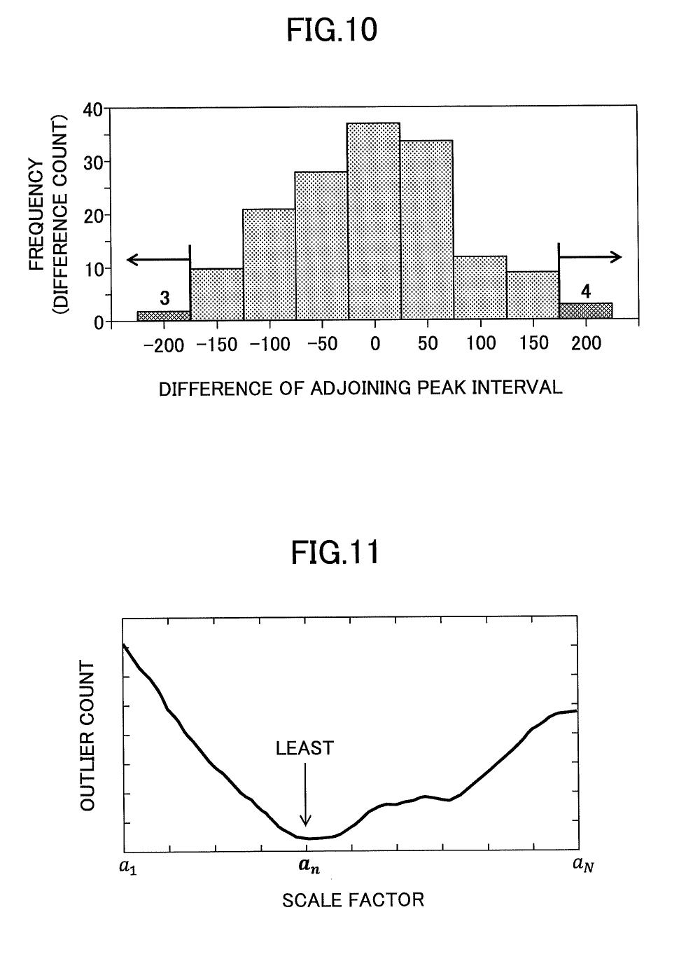

FIG. 10 schematically illustrates a result obtained by the scale factor selector 240 when comparing the data in FIG. 8 with the threshold value on the positive side set to 200 and the threshold value on the negative side set to −200. In FIG. 10, since the frequency greater than or equal to the threshold value on the positive side set to 200 (the number of differences of adjoining peak intervals) is four, and the frequency less than or equal to the threshold value on the negative side set to −200 (the number of differences of adjoining peak intervals) is three, the number of outliers is seven. The scale factor selector 240 executes similar comparison for each of the scale factors, to calculate the number of outliers for each of the scale factors.

Next, at Step S17, the scale factor selector 240 selects a scale factor having a minimum number of outliers as an optimal scale factor. For example, if the numbers of outliers calculated at Step S16 for the scale factors a₁-a_{N} are represented by a graph in FIG. 11, the scale factor selector 240 selects aₙ having the minimum number of outliers as the optimal scale factor. A peak interval of the wavelet coefficient calculated by using the optimal scale factor aₙ is an R-R interval to be obtained.

Note that since the number of peaks detected for each scale factor may vary, it is favorable to normalize the number of outliers. In other words, it is favorable to select a scale factor having a minimum value of [(the number of outliers) divided by (the number of detected peaks)] as an optimal scale factor.

Also, selecting a scale factor having a minimum number of outliers as an optimal scale factor is just an example, and it is not limited as such; a scale factor having minimum variation in differences of adjoining peak intervals may be selected as an optimal scale factor. For example, the scale factor selector 240 may select, from among scale factors, a scale factor having minimum variance or standard deviation in differences of adjoining peak intervals as an optimal scale factor.

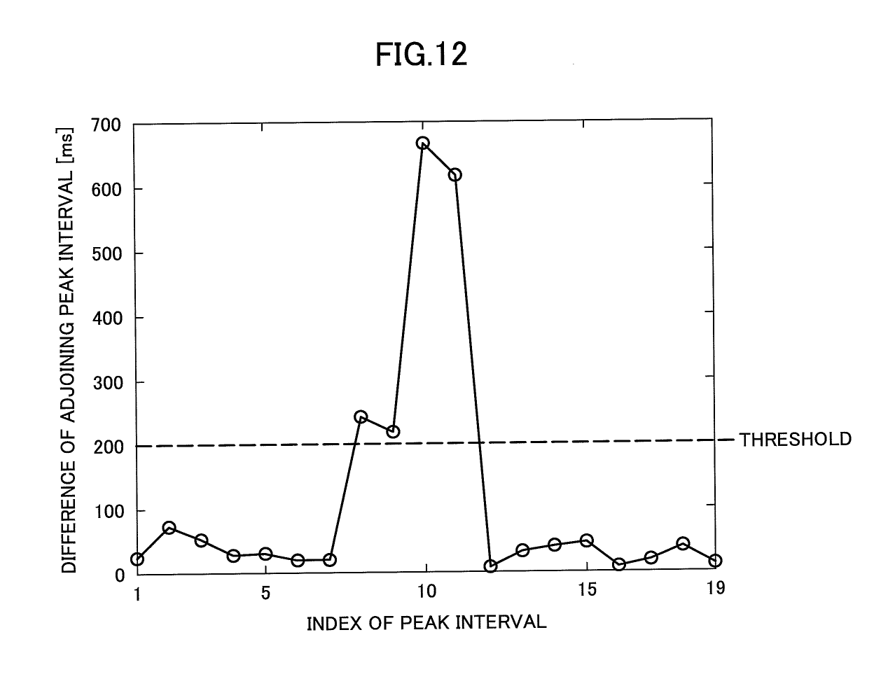

Also, the peak interval calculator 230 may exclude peaks that are not caused by the heartbeat from detection targets when calculating peak intervals. Specifically, the peak interval calculator 230 compares a difference between peak intervals of the wavelet coefficient calculated at Step S17 by using the optimal scale factor “an”, with a predetermined threshold value (a predetermined value), to exclude peaks exceeding the threshold value from detection targets. The predetermined threshold value may be set, for example, to the same value as the threshold value on the positive side described above. With the predetermined threshold value set to 200, for example, in the case where differences of adjoining peak intervals are represented as in FIG. 12, four values exceed the predetermined threshold value, and hence, peaks with which the difference between adjoining peak intervals exceeds the predetermined threshold value are excluded from detection targets.

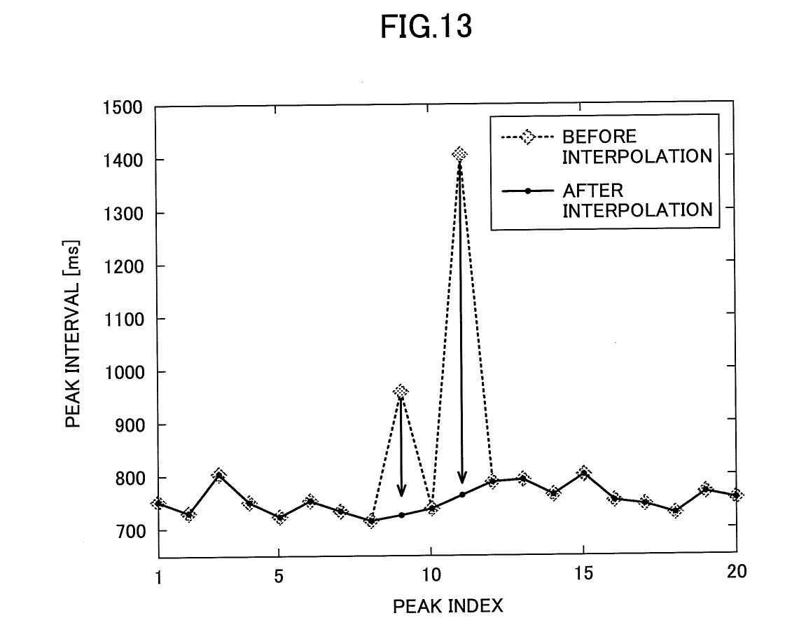

Then, at a time at which a peak exceeding the predetermined threshold is excluded, the peak is interpolated with a calculated value. The peak interval calculator 230 can interpolate a peak, for example, by taking a mean value of peak intervals before and after the excluded one. For example, if peak intervals are represented as in FIG. 13, the peak interval calculator 230 excludes two peaks in dashed-line parts, so as to be interpolated with the mean value of the peak intervals before and after the excluded one (black-dot parts). This makes it possible to further improve precision of R-R intervals to be obtained.

### Application Example

In an application example, an experiment was performed to detect heartbeats by using the heartbeat detection system 1. Particulars of the experiment are presented in Table 1. Note that for comparison, actual heartbeat intervals (R-R intervals) were measured by attaching an electrocardiograph to each subject to obtain a reference, simultaneously with the heartbeat detection by using the heartbeat detection system 1.

TABLE 1

| modulation scheme | unmodulated continuous wave |
|---|---|
| operating frequency | 24 GHz |
| transmission power | 1 mW |
| sampling frequency | 1000 Hz |
| the number of subjects | five persons |
| behavior during measurement | seated, stationary |
| measurement time | 120 sec |
| measurement count | five times |

As an evaluation item 1, detection precision of the R-R intervals was evaluated. Specifically, the RMSE (Root Mean Square Error) was calculated by an expression (1) for R-R intervals measured by the electrocardiograph, and peak intervals measured by the heartbeat detection system 1, to draw a comparison. Note that in the expression (1), N represents the number of detected peaks; tₙ represents a time at which the n-th peak was detected; r(tₙ) represents an R-R interval obtained by the electrocardiograph; and x(tₙ) represents a detected peak interval.

$$RMSE = \sqrt{\frac{1}{N} {\sum }_{n = 1}^{N} \vert r ( {t}_{n} ) - x ( {t}_{n} ) {\vert}^{2}} \qquad ( 1 )$$

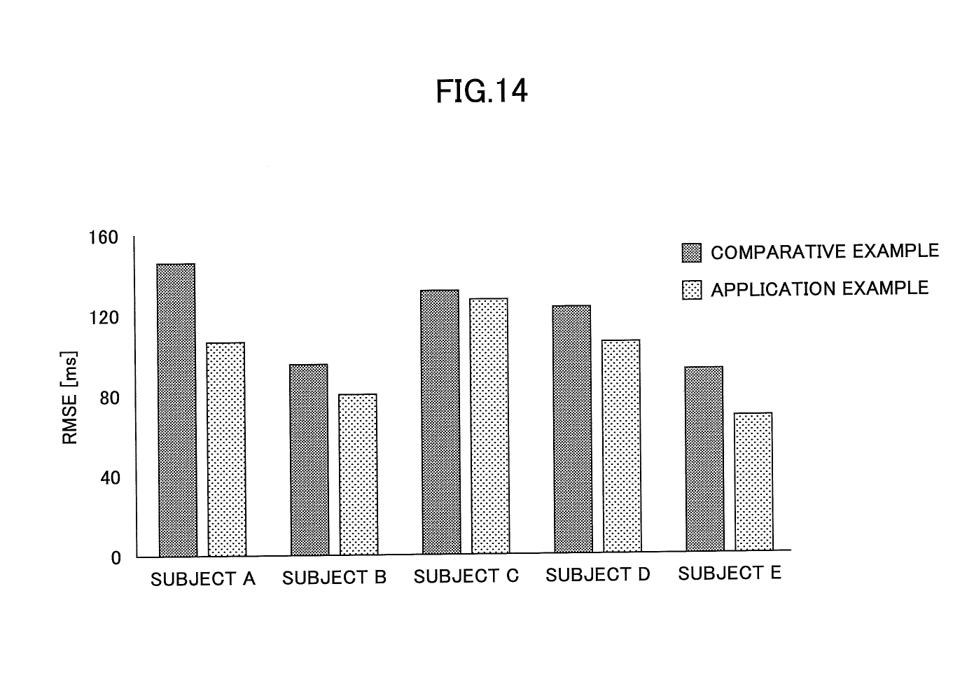

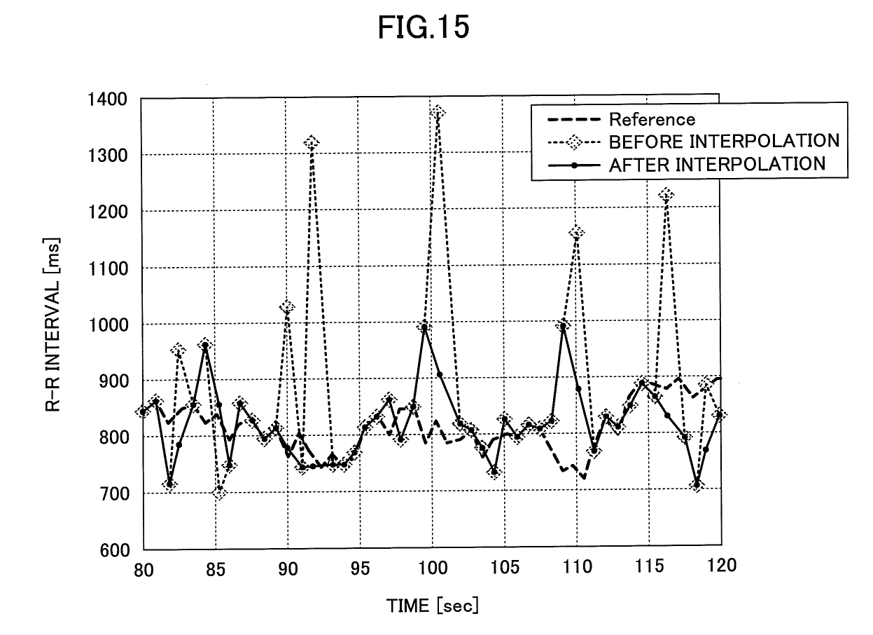

Results of the evaluation item 1 are illustrated in FIG. 14 and FIG. 15. Note that results are also illustrated for a comparative example that uses the technology cited in the background art, which obtains measurement data from a measuring device to measure motion of a human body; stores data, which has been measured for the motion of the human body when a subject as a target of measurement is in a quiet state, as reference data; determines a scale factor based on the reference data; and applies a wavelet transform to the measurement data based on the determined scale factor to measure the heartbeat.

In FIG. 14, a smaller value of the RMSE shows that the measurement was performed closer to the measurement using the electrocardiograph. According to FIG. 14, comparing the application example with the comparative example, heartbeat detection by using the heartbeat detection system 1 exhibited an improved RMSE of R-R intervals for every subject, and the mean value of the improvement of the RMSE was approximately 20 ms.

Also, as illustrated in FIG. 15, comparing data before interpolation with data after interpolation, it was confirmed that detection precision was improved over the entire observation period, by excluding peaks exceeding the predetermined value to be interpolated. In other words, it was confirmed that by removing a peak that is not caused by the heartbeat if detecting such a peak, and by interpolating the peak based on the preceding and succeeding peak intervals, it is possible to detect peaks caused by the heartbeat more precisely. Note that the reference values in FIG. 15 are values measured by the electrocardiograph.

In this way, the heartbeat detection system 1 executes a wavelet transform with multiple scale factors corresponding to heartbeats, to obtain the wavelet coefficient for each of the scale factors. Then, the system calculates, for each of the scale factors, peaks of the wavelet coefficient, each peak interval between peaks next to each other, and further calculates each difference between adjoining peak intervals. Then, the system compares, for each of the scale factors, the calculated differences of adjoining peak intervals, with a threshold value on the positive side and a threshold value on the negative side that have been determined in advance; counts the sum total of a frequency greater than or equal to the threshold value on the positive side, and a frequency less than or equal to the threshold value on the negative side as the number of outliers; and selects a scale factor having a minimum number of outliers as an optimal scale factor.

This makes it possible to calculate peak intervals based on an optimal scale tractor, and hence, enables to calculate peak intervals with higher precision. In other words, R-R intervals can be detected with higher precision. Specifically, the RMSE of R-R intervals can be improved compared with conventional methods.

Also, in the heartbeat detection system 1, the frequency of the heartbeat being approximately 0.5 to 1.2 Hz, and a feature of R-R intervals that intervals next to each other in time do not change significantly, are taken into consideration to select in advance multiple scale factors corresponding to the heartbeat, and to store the scale factors in the RAM 23. Therefore, learning for selecting a scale factor as has been executed in a conventional method becomes unnecessary.

Also, for peak intervals calculated based on an optimal scale factor, differences of adjoining peak intervals may be compared with a predetermined threshold value to exclude peaks exceeding the threshold values from detection targets, and to be interpolated with calculated values (for example, to be interpolated with the mean value of the peak intervals before and after the time of exclusion). This makes it possible to exclude peaks caused by a breath or a small body motion from detection targets of peak intervals, and hence, R-R intervals can be detected with even higher precision.

### Second Embodiment

In a second embodiment, an example of a method of calculating peak intervals will be described in which precision is further raised after selecting a scale factor. Note that in the second embodiment, description of a component that is the same as in the embodiment already described above may be omitted.

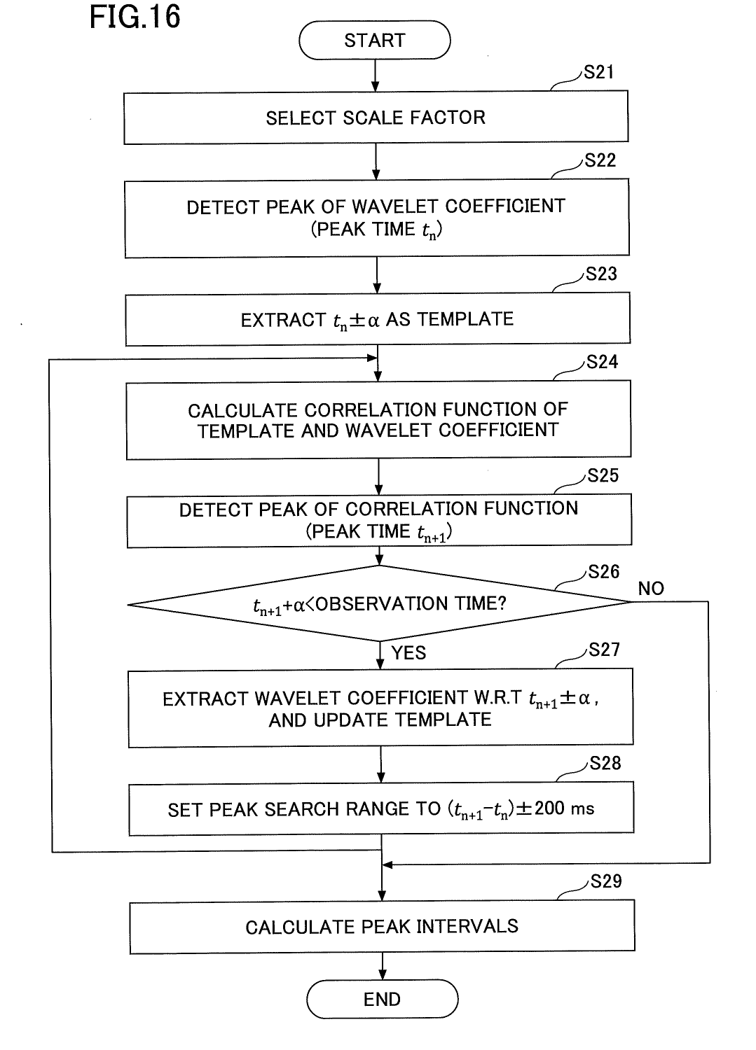

FIG. 16 is an example of a flowchart illustrating operations of a heartbeat detection system according to the second embodiment. Referring to FIG. 16 mainly and the other figures appropriately, a heartbeat detection method according to the second embodiment will be described.

First, at Step S21, the system executes Steps S11 to S17 illustrated in FIG. 5, to select a scale factor.

Next, at Step S22, the peak detector 220 executes a wavelet transform with the scale factor selected at Step S21, to detect peaks of the calculated wavelet coefficient. For example, the maximum R-R interval is set to 1200 ms, to detect a first peak. Note that 1200 ms is just an example, and the maximum R-R interval may be set, for example, based on a statistic of R-R intervals. Here, a peak time tₙ represents a time at which the n-th peak of the wavelet coefficient is detected where N is a natural number, and the time at which the first peak is detected corresponds to the peak time t₁.

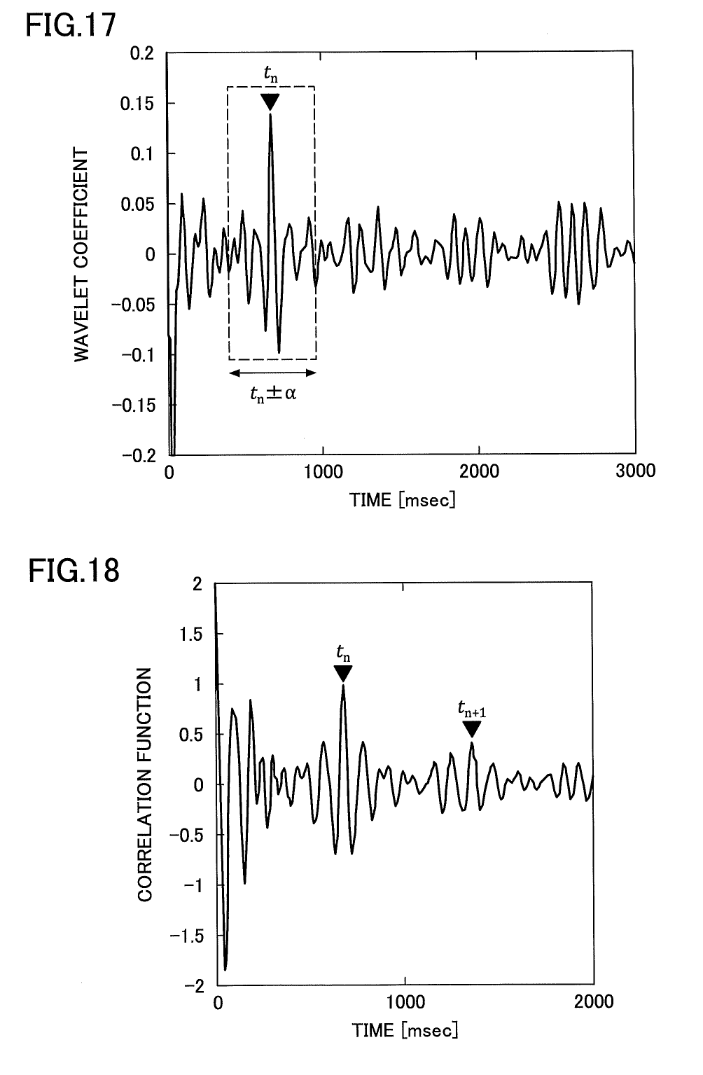

Next, at Step S23, the peak detector 220 extracts tₙ±α as a template. here, α is set to a value such that 2α being the width of the template is greater than or equal to one beat, and less than two beats under an assumed heart rate. Although a specific value of α can be determined by an experiment in advance, it is assumed here that α is set to 300 ms as an example. In other words, 300 ms before and after the peak time tₙ is extracted as the template. The template extracted based on the first peak is t₁±300 ms. FIG. 17 illustrates a peak detected at the peak time tₙ by a reverse triangular mark, and a template designated by a dashed-line frame having the width of tₙ±α.

Next, at Step S24, the peak detector 220 calculates a correlation function of the template and the wavelet coefficient. Here, since the maximum R-R interval is set to 1200 ms, 1200 ms is set as a peak search range, and then, a correlation function is calculated for the wavelet coefficient from t₁ to t₁+1200 ms and the template. FIG. 18 is an example of a correlation function of the template tₙ±α and the wavelet coefficient.

Next, at Step S25, the peak detector 220 detects a peak next to the peak at time tₙ (a peak at time tₙ₊₁) in the correlation function calculated at Step S24. For example, in the correlation function illustrated in FIG. 18, a part designated with a reverse triangular mark of tₙ₊₁ is detected as the next peak. For example, in the case of n=1, the second peak is detected.

Next, at Step S26, the peak detector 220 determines whether tₙ₊₁+α is less than the observation time, namely, determines whether detection has been completed for all peaks. If having determined at Step S26 that tₙ₊₁+α is not less than the observation time (in the case of NO), the peak detector 220 determines that detection has been completed for all peaks, and the process transitions to Step S29. At Step S29, the peak interval calculator 230 calculates peak intervals for all of the detected peaks. These correspond to R-R intervals to be obtained.

If having determined at Step S26 that tₙ₊₁+α is not less than the observation time (in the case of YES), the process transitions to Step S27. At Step S27, the peak interval calculator 230 extracts tₙ₊₁±α as a template, and updates the template. For example, in the case of n=1, it extracts t₂±α as a template and updates the template from t₁±α to t₂±α.

Next, at Step S28, the peak detector 220 sets the peak search range to (tₙ₊₁−tₙ)±200 ms. For example, in the case of n=1, the peak detector 220 sets the peak search range to (t₂−t₁)±200 ms. Here, 200 ms is just an example, which may be set in advance appropriately based on an experiment or the like.

After having completed Step S28, Steps S24 to Step S29 are repeated until it is determined at Step S26 that tₙ₊₁+α is not less than the observation time, and then, the process transitions to Step S29. At Step S29, the peak interval calculator 230 calculates peak intervals for all of the detected peaks as described above. These correspond to R-R intervals to be obtained. Note that every time the process transitions to Step S24, the value of n is increased by one.

In this way, according to this embodiment, a template is extracted from the first detected peak of a wavelet coefficient of an optimal scale factor to search for the next peak, and every time a new peak is detected, the template is updated to detect all the peaks.

Specifically, a template is extracted from a peak (peak time tₙ) of the wavelet coefficient of the optimal scale factor, to calculate a correlation function of the extracted template and the wavelet coefficient, and to detect the next peak (peak at time tₙ₊₁) from the calculated correlation function. Then, a template is newly extracted from the peak at time tₙ₊₁ of the wavelet coefficient and the template is updated, and based on a value of tₙ₊₁−tₙ representing the time interval (interval between peaks next to each other) between the peak time tₙ and the peak time tₙ₊₁, the search range for the next peak is set.

In other words, every time a peak is detected, the template is updated and a peak search range is newly set. Repeating this process can detect the next peak based on a most recently found peak and its immediately preceding peak, and hence, it is possible to raise precision of peak detection.

As above, preferred embodiments have been described. Note that the present invention is not limited to the above embodiments, and various changes and replacements can be applied to the above embodiments without deviating from the scope of the present invention described in the claims.

1. A heartbeat detection system comprising:
a Doppler sensor configured to receive a reflective wave from a subject, to obtain a Doppler signal; and
a processor including a memory and a CPU, configured to execute
applying a wavelet transform to the Doppler signal based on a plurality of scale factors, to obtain a wavelet coefficient for each of the scale factors;
detecting a plurality of peaks of the wavelet coefficient for each of the scale factors;
calculating each peak interval between the peaks next to each other, and each difference between the peak intervals next to each other, for each of the scale factors;
selecting one of the scale factors that has a minimum variation in the differences of the peak intervals, as an optimal scale factor; and
measuring the peak intervals calculated based on the optimal scale factor as heartbeat intervals.

2. The heartbeat detection system as claimed in claim 1, the processor executes
for each of the scale factors, comparing frequency distribution of the differences of the peak intervals next to each other, with a threshold value on a positive side and a threshold value on a negative side that have been determined in advance, and counting a sum total of a frequency greater than or equal to the threshold value on the positive side, and a frequency less than or equal to the threshold value on the negative side, as a number of outliers; and
selecting a scale factor having a minimum number of outliers as the optimal scale factor.

3. The heartbeat detection system as claimed in claim 1, the processor executes selecting, for each of the scale factors, a scale factor having minimum variance or standard deviation in the differences of the peak intervals next to each other, as the optimal scale factor.

4. The heartbeat detection system as claimed in claim 1, the processor executes excluding peaks that are not caused by a heartbeat from detection targets, and calculating the peak interval.

5. The heartbeat detection system as claimed in claim 4, the processor executes
excluding a peak with which the difference between the peak intervals calculated based on the optimal scale factor exceeds a threshold value from detection targets; and
interpolating the peak by taking a mean value of the peak intervals before and after the excluded one.

6. The heartbeat detection system as claimed in any one of claim 1, the processor executes repeatedly extracting a template from a most recently detected peak of the wavelet coefficient of the optimal scale factor to search for a next peak, and updating the template every time the new peak is detected, so as to detect all the peaks.

7. The heartbeat detection system as claimed in claim 6, the processor executes
extracting a template from a peak at a time tₙ of the wavelet coefficient of the optimal scale factor;
calculating a correlation function of the template and the wavelet coefficient of the optimal scale factor;
detecting a next peak at a time tₙ₊₁ from the correlation function;
newly extracting a template from the peak at the time tₙ₊₁ of the wavelet coefficient;
updating the template;
repeating the calculation of a correlation function, the detection of a next peak, and update of a template, so as to detect all the peaks; and
calculating tₙ₊₁−tₙ as the peak interval.

8. The heartbeat detection system as claimed in claim 7, wherein after having updated the template, a search range of the next peak is set based on the value of tₙ₊₁−tₙ.

9. A heartbeat detection method executed by a processor, the method comprising:
obtaining a Doppler signal by a Doppler sensor configured to receive a reflective wave from a subject;
applying a wavelet transform to the Doppler signal based on a plurality of scale factors, to obtain a wavelet coefficient for each of the scale factors;
detecting a plurality of peaks of the wavelet coefficient for each of the scale factors;
calculating each peak interval between the peaks next to each other, and each difference between the peak intervals next to each other, for each of the scale factors;
selecting one of the scale factors that has a minimum variation in the differences of the peak intervals, as an optimal scale factor; and
measuring the peak intervals calculated based on the optimal scale factor as heartbeat intervals.
